(19) United States
(12) Patent Application Publication
(10) Pub. No.: US 2013/0034624 A1
(43) Pub. Date: Feb. 7, 2013

(54) ANIMAL MANAGEMENT

(71) Applicants: Milton Alejandro Gorocica Buenfil, Queretaro (MX); Duane Theuninck, Canyon, TX (US); Todd Raymond Allen, Newton, KS (US); Larry Langford, Wichita, KS (US); Sue K. Denise, Davis, CA (US); David Rosenfeld, San Jose, CA (US); Tom Holm, Salt Lake City, UT (US); Dennis Fantin, Danville, CA (US)

(72) Inventors: Milton Alejandro Gorocica Buenfil, Queretaro (MX); Duane Theuninck, Canyon, TX (US); Todd Raymond Allen, Newton, KS (US); Larry Langford, Wichita, KS (US); Sue K. Denise, Davis, CA (US); David Rosenfeld, San Jose, CA (US); Tom Holm, Salt Lake City, UT (US); Dennis Fantin, Danville, CA (US)

(73) Assignees: CARGILL, INCORPORATED, Wayzata, MN (US); METAMORPHIX, INC., Calverton, MD (US)

(21) Appl. No.: 12/812,491

(22) PCT Filed: Jan. 7, 2009
(86) PCT No.: PCT/US09/30353

§ 371 Date: Mar. 3, 2011

Related U.S. Application Data
(60) Provisional application No. 61/020,249, filed on Jan. 10, 2008.

(51) Int. Cl. C12Q 1/68; A23K 1/18; C40B 30/04

(52) U.S. Cl. 426/2; 435/6.11; 506/9

(57) ABSTRACT

Methods for managing animals can involve identifying a trait by analyzing a biological sample and determining a physical characteristic of a bovine subject using imaging. A method can include obtaining a targeted trait genetic value and determining a targeted trait value for a bovine subject using imaging. A method can include obtaining genetic information determined by identifying, in a biological sample from the first bovine subject, at least one single nucleotide polymorphism (SNP). An animal can be managed based on information that has been identified and/or obtained.

### CLAIM OF PRIORITY AND CROSS-REFERENCE TO RELATED APPLICATIONS

This application claims priority to U.S. provisional patent application 61/020,249 which was filed on Jan. 10, 2008 and is incorporated by reference in its entirety.

### FIELD OF THE INVENTION

The present invention relates to methods and systems for managing animals. More specifically the present invention relates to methods and systems for managing animals by evaluating genetic information and physical characteristic information of the animals.

### REFERENCE TO ELECTRONIC DOCUMENTS CO-FILED WITH APPLICATION

The following electronic files are being filed concurrently herewith:

mmi0201_Table1A.txt: size 9 Mb, created Sep. 10, 2004
mmi0201_Table1B.txt; size 12 Mb, created Sep. 10, 2004
mmi0201_SequenceListing.txt; size 94 Mb, created Jan. 7, 2009

Pursuant to Section 801 of the PCT Instructions Relating to International Applications Containing Large Nucleotide and/or Amino Acid Sequence Listings and/or Tables Relating Thereto, the sequence listing is being filed solely on electronic medium in computer readable form referred to in Section 802. The electronic files in computer readable form are herein incorporated by reference in their entirety.

### BACKGROUND OF THE INVENTION

Under the current standards established by the United States Department of Agriculture (USDA), beef from bulls, steers, and heifers is classified into eight different quality grades. Beginning with the highest and continuing to the lowest, the eight quality grades are prime, choice, select, standard, commercial, utility, cutter and canner. The characteristics which are used to classify beef include age, color, texture, firmness, and marbling, a term which is used to describe the relative amount of intramuscular fat of the beef. Well-marbled beef from bulls, steers, and heifers, i.e., beef that contains substantial amounts of intramuscular fat relative to muscle, tends to be classified as prime or choice; whereas, beef that is not marbled tends to be classified as select. Beef of a higher quality grade is typically sold at higher prices than a lower grade beef. For example, beef that is classified as “prime” or “choice,” typically, is sold at higher prices than beef that is classified into the lower quality grades.

It is known for a cattle processor to pay cattle producers more money for cattle that are expected to provide desirable carcasses. One criterion of a desirable carcass is carcass weight. Another criterion for desirable carcasses is “red meat yield,” or the proportion of saleable beef resulting from a carcass. Red meat yield is negatively correlated to carcass fatness and highly related to a USDA measure known as “yield grade.” Yield grade is measured on a scale from 1 to 5, with 5 being most fat. As cattle get fatter, yield grade value goes up and red meat yield goes down. In most market conditions, yield grade 4 and 5 carcasses are subjected to substantial discounts. Another criterion for desirable carcasses is degree of intramuscular fat, commonly referred to as “marbling.” Marbling is highly related to USDA quality grade. The typical target for marbling is a level associated with USDA Choice. Higher levels of marbling can bring price premiums while lower levels often cause significant price discounts. In general, marbling increases with overall carcass fatness.

Cattle typically arrive at feedlots in heterogeneous groups. It is common for weight of cattle within a pen to vary by 200 lbs or more. During the course of the feeding period, this weight spread tends to increase due to variation in growth rate of individual animals within the pen. There is similar variation in fatness of cattle and carcasses derived from those cattle. It is known and most common within the cattle feeding industry to harvest an entire pen of cattle at the same time. However, this known method of harvesting results in wide variation in resulting carcass weights (and red meat yield, yield grade and marbling) of cattle from the pen.

It is also known to provide a system to calculate an optimum or target condition for an individual cattle and select the individual cattle for shipment based on such calculation. Such known systems typically include the use of ultrasound to determine a characteristic of the cattle (or carcass).

Existing systems typically use the “Cornell Method” for allocating feed to individual animals. The Cornell Method is shown by Fox et al., 1992 Journal of Animal Science 70:3578 and “Application of Ultrasound for Feeding and Finishing Animals: A Review” by P. L. Houghton and L. M. Turlington (Kansas State University, Manhattan 66506). However, this system has several disadvantages including that an optimum or target condition is calculated for an individual cattle and a sorting decision is made for an individual animal based on the calculation.

What is therefore needed are methods and systems to manage animals that integrate more information about each animal to contribute to the categorization and decision making process for populations of animals.

### SUMMARY OF THE INVENTION

The invention relates to methods and systems for managing animals.

In a first aspect, a method for managing animals includes obtaining information that predicts a trait for a first bovine subject of the bovine subjects, at a facility configured for managing bovine subjects. The trait is inferred by analyzing a biological sample of the first bovine subject. The method includes measuring a physiological condition or age of the first bovine subject using internal imaging. The method includes calculating a quantitative score based on at least the genetic information and the determined physical characteristic to predict the trait, and managing the first bovine subject at the facility based on the quantitative score. The method includes managing the first bovine subject at the facility based on at least the predicted trait based on the quantitative score.

Implementations can optionally include the following features. The method can further include obtaining, at the facility, the biological sample from the first bovine subject; and forwarding the biological sample to a laboratory to perform the analysis of the biological sample, wherein the information is obtained at the facility from the laboratory. The information can be received electronically at the facility from the laboratory. Managing the first bovine subject can include performing an operation selected from the group consisting of: selecting the first bovine subject for harvest, selecting the first bovine subject for being relocated, selecting the first bovine subject for receiving treatment, selecting the first bovine subject for being measured, grouping the first bovine subject with at least another of the bovine subjects, and combinations thereof. The identified trait can be at least one characteristic selected from the group consisting of: an average daily weight gain for the first bovine subject, a red meat yield of the first bovine subject, a tenderness of the first bovine subject, an endpoint characteristic of the first bovine subject, a ribeye area of the first bovine subject, and a marbling of the first bovine subject. The identified trait that is taken into account in managing the first bovine subject can include at least the marbling of the first bovine subject, and the method can further include making an implant decision regarding the first bovine subject based on at least the average daily weight gain for the first bovine subject and the marbling of the first bovine subject. The analysis of the biological sample can include identifying a nucleotide occurrence of at least one single nucleotide polymorphism (SNP) corresponding to a position that is about 500,000 or less nucleotides from position 300 of at least one of SEQ ID NOS:19473 to 21982, wherein the SNP is associated with the trait. The analysis of the biological sample can include identifying a nucleotide occurrence of a panel of SNPs. The analysis of the biological sample can further include contacting a bovine polynucleotide in the biological sample with an oligonucleotide that binds to a target region of any one of SEQ ID NOS:24493 to 64886, wherein the target region comprises a position corresponding to position 300 of any one of SEQ ID NOS:19473 to 21982 or wherein the target region is within 3000 nucleotides of a nucleotide corresponding to position 300 of any one of SEQ ID NOS:19473 to 21982. The determined physical characteristic can be at least one characteristic selected from the group consisting of: a marbling of the first bovine subject, a backfat measurement of the first bovine subject, a muscle depth measurement of the first bovine subject, and combinations thereof. The facility can include an animal management location at which the bovine subjects are to be kept for a yet undetermined time period before being removed therefrom at a shipping date, and the method can further include receiving the bovine subjects including the first bovine subject at the animal management location, the bovine subjects being organized in several arrival groups; and generating a future backfat estimate for the first bovine subject; wherein managing the first bovine subject comprises sorting, based on at least the future backfat estimate and the identified trait, the first bovine subject into one of several predetermined sort groups for separate management at the animal management location, the predetermined sort groups being different from the arrival groups and associated with different predefined shipping dates. The facility can be a feedlot where the bovine subjects including the first bovine subject are managed.

In a second aspect, a method for managing animals includes obtaining a targeted trait genetic value for a first bovine subject based on analysis of a biological sample of the first bovine subject. The method further includes determining a targeted trait value for the first bovine subject using imaging. The method further includes sorting the first bovine subject into one of multiple predefined groups based on at least the obtained targeted trait genetic value and the determined targeted trait value.

Embodiments can optionally include the following features. The targeted trait genetic value can include a marbling genetic value, and the method can further include determining a marbling score using the obtained targeted trait genetic value, the determined targeted trait value, an implant dose value for the first bovine subject and a target backfat value for the first bovine subject, wherein the sorting is done based on the marbling score. The method can further include selecting one of multiple time categories for the first bovine subject, the categories including at least an early time category, a normal time category, and an extended time category; and determining the marbling score more than once for the first bovine subject while varying at least one of the implant dose value and the target backfat value, the variation being defined in a schedule associated with the selected time category. The method can further include selecting an actual implant dose for the first bovine subject based at least on: (i) the targeted trait genetic value; and (ii) a genetic value relating to an average daily weight gain for the first bovine subject, the genetic value based on analysis of a biological sample of the first bovine subject. The analysis of the biological sample can include identifying a nucleotide occurrence of at least one single nucleotide polymorphism (SNP) corresponding to position 300 of at least one of SEQ ID NOS:19473 to 21982, wherein the SNP is associated with a marbling trait of the first bovine subject.

In a third aspect, a method for managing animals includes obtaining genetic information regarding a first bovine subject, the genetic information determined by identifying, in a biological sample from the first bovine subject, at least one single nucleotide polymorphism (SNP). The method includes generating a physical attribute estimate for the first bovine subject using at least one physical measurement of the first bovine subject and an equation configured to make estimations for a single animal. The method includes managing the first bovine subject based on the genetic information and the physical attribute estimate.

Embodiments can include any, all or none of the following features. Managing can include sorting the first bovine subject into one of several predetermined sort groups for separate management at an animal management location, wherein the predetermined sort groups are different from arrival groups and are associated with different predefined shipping dates. The at least one SNP can correspond to a position that is about 500,000 or less nucleotides from position 300 of at least one of SEQ ID NOS:19473 to 21982. The genetic information can further be determined by contacting a bovine polynucleotide in the biological sample with an oligonucleotide that binds to a target region of any one of SEQ ID NOS:24493 to 64886, wherein the target region comprises a position corresponding to position 300 of any one of SEQ ID NOS:19473 to 21982 or wherein the target region is within 3000 nucleotides of a nucleotide corresponding to position 300 of any one of SEQ ID NOS:19473 to 21982. The physical measurement can be an imaging measurement of the first bovine subject. The method can further include estimating an empty body fat measure for the first bovine subject using the imaging measurement; determining a feed allocation using a predefined algorithm taking into account the estimated empty body fat measure; and administering feed according to the determined feed allocation. The physical attribute estimate can include a future weight estimate based at least in part on an estimated daily-gain-to-finish measure for the first bovine subject, the estimated daily-gain-to-finish measure also being directly used in managing the first bovine subject; and wherein the managing is also based on an estimated days-to-critical-weight measure for the first bovine subject, the days-to-critical-weight measure being estimated using at least an estimated daily-gain-to-finish measure for the first bovine subject and a predefined critical weight for animals.

Embodiments can provide any, all, or none of the following advantages. Improved animal management can be provided. A genetic trait and a body characteristic of a bovine subject can be taken into account to provide more efficient animal management. More cost-efficient sorting procedures, for example at animal feedlots, can be provided. The economic process of deriving profit from a bovine subject can be streamlined and made more effective. Measures taken in animal management can be better adapted to individual bovine subjects by taking into account one or more genetic factors.

In a fourth aspect, a method for predicting a trait in an animal subject includes analyzing genetic information in a biological sample of the animal subject; and determining a physical characteristic of the animal subject using imaging; and calculating a quantitative score based on at least the genetic information and the determined physical characteristic to predict the trait.

In a fifth aspect, a system for use in managing animals includes a management component, a measurement component and a genetic component.

The details of one or more embodiments are set forth in the accompanying drawings and the description below. Other features and advantages will be apparent from the description and drawings, and from the claims.

### DESCRIPTION OF DRAWINGS

FIG. 1 schematically shows an example of an animal management system.

FIG. 2 schematically shows exemplary measurement components of an animal management system.

FIG. 3 shows an exemplary top-view of making an ultrasound measurement using the animal management system of FIG. 1.

FIG. 4 schematically shows exemplary management components of an animal management system.

FIG. 5 schematically shows an exemplary genetics component of an animal management system.

Like reference symbols in the various drawings indicate like elements.

### DETAILED DESCRIPTION

This application relates to the following applications:

Ser. No. 10/750,185, filed Dec. 31, 2003 and entitled “Compositions for inferring bovine traits”;

Ser. No. 10/750,622, filed Dec. 31, 2003 and entitled “Compositions, methods and systems for inferring bovine breed”;

Ser. No. 10/750,623, filed Dec. 31, 2003 and entitled “Methods and systems for inferring bovine traits”;

Ser. No. 60/437,482, filed Dec. 31, 2002 and entitled “Methods and systems for inferring genetic traits to manage bovine livestock”, to which each of the previously mentioned applications claims priority;

Ser. No. 60/631,469, filed Nov. 29, 2004 and entitled “Animal Management System”; and

International Application Number PCT/US2005/043069, filed Nov. 29, 2005 and entitled “Animal management system”.

The entire contents of each of the above applications is hereby incorporated by reference.

The specification hereby incorporates by reference in their entirety, the electronic documents filed herewith. The electronic files are titled “MMI0201WP Table 1A.doc” which is 11 Mb in size, and a file called “MMI0201WP Table 1B.doc” which is 11 Mb in size, both created Jan. 6, 2009 and a file called “14972-105021 MMI-0201lfr_ST25.txt” which is 94 Mb in size created on Jan. 7, 2009.

Systems and methods are described for use in managing animals using genetic information, and information about the physical characteristics of animals. These systems and methods are used to obtain information regarding a trait of an animal and then used to sort and manage animals effectively. “Animal,” “animals” or “livestock” generally refer to any number of domesticated and/or wild animals such as swine, cattle, horses, bison, goats, sheep, deer, elk, alpaca, llama, poultry animals, fish, etc. As used herein, the term “trait” refers to a measured or observed characteristic of an animal at a particular age or condition in an animal's life cycle. The trait may also be predicted for a particular timepoint or outcome such as time of harvest.

In one embodiment, animals at a feedlot are sorted based on genetic information or physical characteristics that are imaged using imaging methods. In one embodiment, management decisions are made to keep, breed, cultivate or cull an animal. Animals that are cultivated are optionally provided additional factors to optimize growth and development of the animal. Such factors may be, but are not limited to, alternate feed, presence of feed additives, presence of implants, variations in dose of implanted molecule or compound, or combinations thereof. Information provided to feedlot operators can be used to change the subsequent treatment of individual animals at the feedlot, such as to increase or decrease feed, or to administer certain materials, such as growth factors.

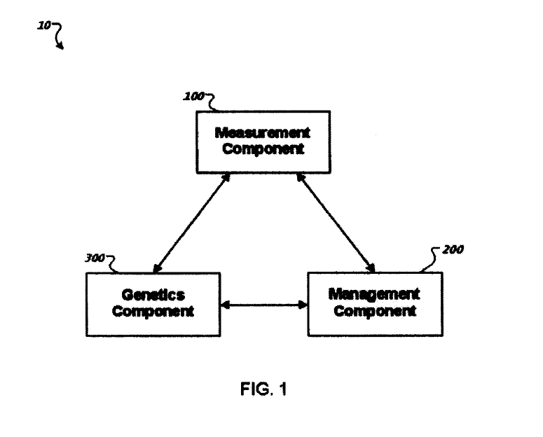

FIG. 1 shows a high-level representation of an exemplary system 10 for managing animals at a facility, for example at a feedlot. The system 10 includes a measurement component 100 and a management component 200 that can be in communication with each other and/or in communication with a genetics component 300. In one embodiment, the genetics component 300 and the measurement component 100 provide information to the management component 200 for use in decision making for animal management.

The measurement component 100 includes, for example, measurement equipment for performing physical characteristic measurements on a given animal. Measurements may include external physical characteristics such as length, weight and the like. As used herein, the term physical characteristic means an objective measurement of a physiological condition at the time of measurement. Measurement may also include internal physical characteristics such as, but not limited to, backfat thickness, muscle tissue depth, amount of marbling, ribeye area, follicular development, bone development, and tenderness that may be imaged using conventional internal imaging methods. Conventional imaging techniques include, but are not limited to two dimensional (2D) imaging, three dimensional (3D) imaging, computed tomography (CT), magnetic resonance imaging (MRI), x-ray or radiation, positron emission tomography (PET), single photon emission computerized tomography (SPECT), computerized tomosynthesis (CT), ultrasound (US), angiographic, fluoroscopic, visual light photography, infrared photography, and the like or combination thereof. Of these techniques, ultrasonography is the least expensive and is particularly well suited to use on large animals raised for commercial food production. In one embodiment, the values from imaging are used in combination with the genetics component to manage animals.

Ultrasound imaging involves the direct introduction of high frequency sound waves from a transducer into the tissue to be evaluated. The echo resulting from these sound waves can be recorded as an image that provides valuable information about the internal characteristics of the tissue. The time delay between transmitting the sound waves and recording the echo can be used to indicate the depth of the tissue being imaged. The intensity of the echo can be used to distinguish between different types of tissue, because different materials have different levels of acoustical impedance. In this way, internal structures can be visualized, including overall organs and structures on or within organs, such as lesions.

In specific embodiments, one or more characteristics such as backfat thickness, muscle tissue depth, follicular development or an amount of marbling can be determined using a handheld ultrasound probe. In another embodiment, near infrared reflectance may be used to measure tenderness of a bovine subject. In still another embodiment, x-ray may be used to measure bone development of a bovine subject.

The data obtained by ultrasound tissue imaging and analysis at a packing plant is itself indicative of meat quality and/or yield, such as the backfat measurements, or can be used to make other calculations, such as yield grade. Yield grade is a scale from 1 to 5, with 1 being the most lean and 5 the least lean.

Typically, cattle backfat thickness varies from about 0.1 inch to about 1.0 inch thick. Rib eye area typically varies from about 9 square inches to about 15 square inches. Yield grade is determined by considering at least rib eye area and backfat. First though, solely with respect to backfat, backfat measuring greater than about 0.7 inch thick generally results in a yield grade of 4 or better. Average cattle have a backfat thickness ranging from about 0.4 inch to about 0.7 inch, and such backfat generally results in a yield grade of 3. Less backfat results in a yield grade of 1-2.

Yield grade also considers rib eye area. The USDA yield grade is determined by considering backfat thickness, rib eye area, hot carcass weight (which is determined by weighing both halves of a carcass about 15 minutes after initial processing) and pelvic, kidney and heart fat (PKH) values. Thus, for example, if a particular animal has a relatively small rib eye area and relatively thick backfat, then the animal likely will receive a yield grade of 4 or 5. And, if the animal has relatively large rib-eye area and relatively little backfat, then the animal likely would receive a yield grade of 1-2.

Marbling also can be determined using ultrasound tissue imaging and analysis of ruminants at packing plants. Marbling is determined by computer analysis of contrast differences in the ultrasound image. A quality grade is then assigned to the animal to reflect the marbling content. Marbling is specified as standard (which correlates with the least amount of marbling), select, choice and prime (prime correlates with the most amount of marbling).

The measurement component can also store such information measured from one or more live animals.

The genetics component 300 includes equipment for obtaining genetic information about a given animal, such as by taking a biological sample, causing it to be analyzed, and providing a result of the analysis. Thereafter, the genetics component can also store such information (e.g., information about a live animal's genome that can relate to physical traits, or phenotypes such as marbling, average daily weight gain, red meat yield, tenderness, fat thickness, and the like) determined from one or more live animals.

One or ordinary skill in the art will appreciate that in some embodiments the physical characteristic measured in the measurement component 100 may be the same as the trait that information is determined for by the genetics component 300. In other embodiments, the physical characteristic measured by the measurement component 100 is different than the trait information determined by the genetics component 300.

Biological samples can be harvested from a live animal or an animal carcass. Suitable biological samples include tissue or fluid suspected of containing a polynucleotide from an individual. Such biological samples include, but are not limited to whole blood, plasma, serum, spinal fluid, lymph fluid, the external sections of the skin, respiratory, intestinal, and genitourinary tracts, tears, saliva, blood cells, tumors, organs, milk, semen, tissue and samples of in vitro cell culture constituents. In some embodiments, a biological sample useful for inferring traits about an individual animal, such as a bovine subject, can come from a biological sample of that animal (e.g., that contains nucleic acid molecules, including portions of the gene sequences to be examined, or corresponding encoded polypeptides, depending on the particular method used to analyze the biological sample). A biological sample useful for practicing a method of the invention can be one containing deoxyribonucleic (DNA) acid or ribonucleic acids (RNA). The biological sample generally can contain a deoxyribonucleic acid sample, particularly genomic DNA or an amplification product thereof. However, where heteronuclear ribonucleic acid which includes unspliced mRNA precursor RNA molecules and non-coding regulatory molecules such as RNA is available, a cDNA or amplification product thereof can be used. In some embodiments, the biological sample 124 can be obtained from a live animal at, or near, the time of arrival through the use of a tool that substantially simultaneously collects a tissue sample from the animal while placing an identifying ear tag. In other embodiments, the biological sample can be obtained by, for example, collecting a whole blood sample.

In some embodiments, the biological sample can then be analyzed on the premises, or be sent out to a laboratory where a genotyping system can be used to analyze the sample. Exemplary genotype systems typically includes a hybridization medium and/or substrate that includes at least two oligonucleotides of the present invention, or oligonucleotides used in the methods of the present invention. For example, a solid support can be provided, to which a series of oligonucleotides can be directly or indirectly attached. In another aspect, a homogeneous assay is included in the system. In another aspect, a microfluidic device is included in the system. The hybridization medium or substrates are used to determine the nucleotide occurrence of single nucleotide polymorphism (SNP) markers that are associated with a trait.

Accordingly, the oligonucleotides are used to determine the nucleotide occurrence of bovine SNPs that are associated with a trait. The determination can be made by selecting oligonucleotides that bind at or near a genomic location of each SNP of the series of bovine SNPs. The system of the present invention typically includes a reagent handling mechanism that can be used to apply a reagent, typically a liquid, to the solid support. The binding of an oligonucleotide of the series of oligonucleotides to a polynucleotide isolated from a genome can be affected by the nucleotide occurrence of the SNP. The system can include a mechanism effective for moving a solid support and a detection mechanism. The detection method detects binding or tagging of the oligonucleotides.

Medium to high-throughput systems for analyzing SNPs, known in the art such as the SNPStream® UHT Genotyping System (Beckman/Coulter, Fullerton, Calif.) (Boyce-Jacino and Goelet Patents), the Mass Array™ system (Sequenom, San Diego, Calif.) (Storm, N. et al. (2002) Methods in Molecular Biology. 212: 241-262.), the BeadArray™ SNP genotyping system available from Illumina (San Diego, Calif.)(Oliphant, A., et al. (June 2002) (supplement to Biotechniques), and TaqMan™ (Applied Biosystems, Foster City, Calif.) can be used with the present invention. However, the present invention provides a medium to high-throughput system that is designed to detect nucleotide occurrences of bovine SNPs, or a series of bovine SNPs. Therefore, as indicated above the system includes a solid support or other method to which a series of oligonucleotides can be associated that are used to determine a nucleotide occurrence of a SNP for a series of bovine SNPs that are associated with a trait. The system can further include a detection mechanism for detecting binding of the series of oligonucleotides to the series of SNPs. Such detection mechanisms are known in the art.

These systems can be used, for example, to identify a nucleotide occurrence of a panel of SNPs corresponding to positions that are about 500,000 or less nucleotides from position 300 of at least one of SEQ ID NOS:19473 to 21982, where the SNP is associated with a trait such as marbling or average daily gain.

In one embodiment, the present invention provides an isolated polynucleotide that includes a fragment of at least 20 contiguous nucleotides of the bovine genome, or a complement thereof, where the isolated polynucleotide includes a nucleotide occurrence of a SNP associated with a trait, where the SNP is in disequilibrium with a SNP corresponding to position 300 of any one of SEQ ID NOS:19473 to 21982. In certain aspects, the polynucleotide is located about 500,000 or less nucleotides from position 300 of SEQ ID NOS:19473 to 21982 on the bovine genome. The linkage disequilibrium for cattle is about 500,000 nucleotides. Therefore, it is expected that other SNPs can be identified that are associated with the same traits based on the fact that these other SNPs are located less than or equal to about 500,000 nucleotides of the identified associated SNP on the bovine genome. In certain aspects, the polynucleotide is from an Angus, Charolais, Limousin, Hereford, Brahman, Simmental or Gelbvieh bovine subject.

In certain aspects, the isolated polynucleotide includes a fragment of at least 5, 10, 15, 20, 25, 30, 35, 40, 45, 50, 100, 200, 250, 500, 1000, 5000, 10,000, 25,000, 50,000, 100,000, 125,000, 250,000 or 500,000 nucleotides in length. Furthermore, in certain examples, the isolated polynucleotide includes a fragment of at least 5, 10, 15, 20, 25, 30, 35, 40, 45, 50, 100, 200, 250, 500, 1000, 5000, or 9549 contiguous nucleotides of any one of SEQ ID NOS:24493 to 64886. In another aspect, the isolated polynucleotide is at least 65, 70, 75, 80, 85, 90, 95, 96, 97, 98, 99, or 99.5% identical to the recited sequences, for example. In another aspect, the isolated nucleotide includes region that is contiguous with a region of any one of SEQ ID NOS:19473 to 21982 that includes position 300. In certain aspects, the isolated polynucleotide consists of any one of SEQ ID NOS:19473 to 21982. In other aspects, the isolated polynucleotide consists of any one of SEQ ID NOS:21983 to 24492. In certain aspects, isolated polynucleotides include an associated SNP or are complementary to a region of at least 20 contiguous nucleotides that includes an associated SNP. Accordingly, in these aspects the isolated polynucleotide includes a nucleotide at position 300 of any one of SEQ ID NOS:19473 to 21982.

The analysis of the biological sample can also include contacting a bovine polynucleotide in the biological sample with an oligonucleotide that binds to a target region of any one of SEQ ID NOS:24493 to 64886, wherein the target region comprises a position at position 300 of any one of SEQ ID NOS:19473 to 21982 or wherein the target region is within 3000 nucleotides of a nucleotide at position 300 of any one of SEQ ID NOS:19473 to 21982. The attached sequence listing provides sequences of contigs (SEQ ID NOS:24493 to 64886) generated from the bovine genome. It will be understood that contigs can be aligned such that SNPs that are on separate contigs, but are located within 500,000 nucleotides on the bovine genome, can be identified. For example, alignment of contigs and determination of distance between contigs provided herein, can be accomplished by using the sequence information of the human genome as a scaffold. Tables 1A and 1B (filed herewith on the compact disc), lists contigs that are “nearby” (i.e. within 500,000 nucleotides on the bovine genome) an associated SNP. While two methods have been described here for using biological samples to infer traits, other methods can be used to identify certain genetic information that has been previously determined to correlate to traits.

Tables 1A and 1B, both of which are filed herewith on a compact disc, disclose the SNPs identified by the analysis, and provide the SNP names for the SNPs corresponding to position 300 of SEQ ID NOS:19473 to 21982. The sequences disclosed in SEQ ID NOS:SNP1 to SNP4000 are regions from which amplicons were generated. Table 1B also indicates the location of the amplicon-generating regions within a larger bovine genomic sequence contig (SEQ ID NOS:24493 to 64886) (See column 2 of Table 1B, labeled “In Sequence,” which lists a contig name (e.g., “19866880525139”) and positions (e.g. “923-1522”) within the contig of an amplicon which includes the SNP at position 300. A sequence identifier for the amplicon (SEQ ID NOS:19473-21982) is listed in Table 1A. Furthermore, Tables 1A and 1B identify the nucleotide occurrences that have been detected for each of these SNPs, and identifies traits that have been identified to be associated with these SNPs using methods disclosed herein. All of the SNPs listed in Tables 1A and 1B were associated with the respective trait(s) with a confidence level of 0.01, or higher confidence. Finally, Table 1A provides the sequence of an extension primer that was used to determine the nucleotide occurrence of the SNPs (SEQ ID NOS:21983 to 24492).

Each SNP in Tables 1A and 1B is characterized by the trait(s) found to be in association: marbling, tenderness, fat thickness, daily gain and retail yield. For each of the five traits, “High” refers to the direction of the specific allele contributing to the largest value of the trait. “Low” refers to the direction of the specific allele contributing to the smallest value of the trait. Because each SNP marker is represented by two copies of nucleotides, each animal can have two copies, one copy or no copies of the specified allele. For example, a SNP with two alleles: guanine (G) and cytosine (C) can produce three possible genotypes: GG, GC and CC. If animals with the GG genotype have significantly higher means than animals with the CC genotype, then the G SNP contributes “high” and the C SNP contributes “low”.

In certain aspects of the invention directed at methods for inferring traits such as the traits listed in Tables 1A and 1B, nucleotide occurrences are determined for one or more associated SNPs. Therefore, in one aspect, for example, the method is used to infer fat thickness, by determining a nucleotide occurrence of at least one SNP corresponding to the SNPs indicated in Tables 1A and 1B as associated with fat thickness. For this aspect, as a non-limiting example, a nucleotide occurrence of the SNP at position 300 of SEQ ID NO:19473 can be identified and compared to the nucleotide occurrences listed in Tables 1A and 1B 1 for SEQ ID NO:19473. A thymidine residue at position 300 of SEQ ID NO:19473 infers a higher likelihood that the bovine subject will produce meat that has high tenderness. In addition, as a non-limiting example, a nucleotide occurrence at position 300 of SEQ ID NO:19474 can be determined and used alone or in combination with the nucleotide occurrence at position 300 of SEQ ID NO:19473, to infer tenderness. For example, if position 300 of both SEQ ID NO:19473 and SEQ ID NO:19474 are thymidine residues, there is an even greater likelihood that the bovine subject will produce meat that has high tenderness, than for either nucleotide occurrence alone.

In some embodiments, the system 10 can perform a method for managing animals in which the genetics component 300 obtains a targeted trait genetic value (e.g., a marbling genetic value, a red meat yield genetic value, a tenderness genetic value, or an average daily gain, etc) for a first bovine subject based on analysis of a biological sample of the first bovine subject. Generally a panel of single nucleotide polymorphism (SNP) markers is used to calculate a targeted trait genetic value. In some embodiments, the panel of markers may include at least 2, 3, 4, 5, 7, 15, 20, 25, 50, 75, or 100 SNP markers.

Genetic information from genes, genotypes, alleles or DNA markers can be used independently or combined from a number of genes, genotypes, alleles or DNA markers into a single composite genetic value or score. In some embodiments, individual genotypic information from individual genes, alleles, genotypes or markers can be used in a regression analysis within or across breeds. Animals can then be ranked according to their regression scores or actual values assigned to animals based on their individual scores. In some embodiments, genes, alleles, genotypes or markers can be combined as in a molecular breeding value or molecular genetic value. The composite value is a prediction of the genetic potential based on their genetic data individual contribution to the physical characteristic of the animal. In other embodiments, genetic information from genes, genotypes, alleles or DNA markers can be combined with an animal's correlated physical characteristics and physical characteristics from related animals to predict total genetic merit. An example of this strategy would incorporate genes, genotypes, alleles or DNA markers into genetic prediction models that rely on phenotypes of related individuals, such as those that estimate expected progeny differences or predicted differences or estimated breeding values.

In one embodiment, a composite genetic information score from multiple genes, genotypes, alleles or DNA markers is a Molecular Genetic Value. A Molecular Genetic Value, or MGV, is a mathematical expression developed to help explain and use the genetic results from quantitative trait analysis. Examples of commercial products include Tru-Marbling™ and Tru-Tenderness™ tests (MMI Genomics, Davis, Calif.). Traits such as marbling, tenderness, growth and many others are classified as “quantitative” traits because their expression is controlled by a large number of genes rather than single genes. DNA-based diagnostic tests containing many genes and DNA markers have been developed and validated for certain traits. The MGV is a method for reporting the combined effects from a large set of DNA markers into a single expression that is easy to interpret and utilize for enhancing breeding and selection decisions.

In order to combine information from a number of DNA markers, the genetic effect of each genotype included in the MGV diagnostic test must be partitioned into additive and non-additive components. A research population of animals from which genotypes have been determined and individual trait phenotypes recorded is used to estimate these effects using mathematical models. In one embodiment, a Bayesian strategy can be employed using a Markov Chain Monte Carlo approach. This statistical algorithm partitions a phenotype into components described by Falconer (and universally accepted by quantitative geneticists) due to genetic (as defined by molecular markers), known environmental effects (like sex, feedlot, etc) and residual variation. This algorithm uses an iterative strategy to find the best fit of the genotypic data to the phenotypic data. The same hierarchy of models that were tested in the classical setting can be used, except that all SNPs can be now included in the one analysis. A suitable variable selection procedure can be included in the Bayesian regression set up in order to identify models with the highest posterior probability. This strategy is unique because it analyzes all markers in a single analysis.

Upon completion of the statistical analysis, estimates of the magnitude of the effect of all SNP markers in the diagnostic for all possible genotypes of a SNP in the research population are available. These data are used to create a table of values in the database that has marker genotype and contribution to the phenotype. For example, GG may have a value of 0.05, GC may have a value of 0.01 and CC may have a value of −0.05 at a specific SNP. Epistasis is the interaction among different loci, so if two markers interact, this analysis provides a table of values for all 9 combinations of the genotypes of the two markers.

Results are stored in a database and used to predict trait values of animals with unknown phenotypes using the Molecular Genetic Value (MGV). Animals for which prediction of trait values are desired are genotyped utilizing previously described platforms, then the genotypes are compared to the database of individual marker values. These marker values for each SNP genotype in the diagnostic test are summed across all diagnostic markers creating the MGV for an individual animal. Animals with MGVs greater than 0 are predicted to have trait values greater than the mean and the relative deviation from the mean is proportional to the degree by which the MGV is different from 0. Likewise, animals with MGV less than 0 are predicted to have MGVs less than the mean and the relative deviation from the mean is proportional to the degree by which the MGV is less than 0. Animals with an MGV near 0 are predicted to perform near the mean value of the trait.

Molecular Genetic Values can be used to enhance animal breeding and selection decisions in a number of important ways. MGVs can be used to rank animals based on their genetic potential to express a trait. These ranking can then be utilized in comparing one animal against any other animal so that decisions can be made on whether to keep, breed, flush, cull or sell specific animals. MGVs can be used to enhance mating decisions. Software tools can provide probability outcomes for MGVs of progeny produced from specific matings to previously tested sires and dams. These breeding tools can be used to optimize the MGVs from progeny produced in defined matings. Since the MGV for an animal will not change over time and since it is based on the animal's actual DNA genotype, the MGV can be used to make early and accurate breeding decisions. The ability to rank and breed animals at an early age results in both increasing the accuracy of selection and decreasing the age at which animals can be selected.

In this animal management system 10, the measurement component 100 and the genetic component 300 can be in communication with the management component 200 such that the management component 200 can use information from the measurement component 100 and/or the genetics component 300 to provide animal management, such as sorting decisions, time of harvest decisions, implant decisions (e.g., decisions regarding administration of medicaments and/or compositions which can be intended, for example, to cause the individual animals to grow faster, grow more efficiently, and/or produce leaner carcasses), commingling of cattle at the time of sorting, and allocating feed provided to a pen to individual animals within the pen. In some embodiments, a quantitative score can be calculated for a trait using an equation that includes information from the genetics component and measurement component. The score outputted from this equation is used to manage a group of animals.

Equations for obtaining such quantitative scores are developed using multiple regression tools. Predictive variables that are evaluated include items such as weight, ultrasound measurements of backfat, marbling and muscle depth, live animal estimates of empty body fat, MGV value, implant dose, expected values for backfat and empty body fat at slaughter. The general approach is to use stepwise regression on these predictive variables and their interactions in order to screen for variables with greatest predictive value. After the initial screening, standard methods for multiple regression and residual analysis were used to derive working equations. In one embodiment, the equation that is used for this method can be generally presented as:

Trait Score=constant+(X*MGV for the trait)+(Y*imaged characteristic)+(Z*additional factors)

In general, the system 10 can use a combination of one or more physical measurements, imaging results, and/or one or more portions of genetic information of a specific live animal to predict future characteristics such as weight, body composition, and marbling. These factors can then be taken into account for management decisions such as in sorting and harvest date decisions.

Traits that can be assigned a score using this method generally include those traits that have a genetic component, and are associated with a physical characteristic that can be measured externally or using imaging techniques. Examples of such traits include, but are not limited to marbling, tenderness, quality grade, quality yield, muscle content, fat thickness, feed efficiency, red meat yield, average daily weight gain, feed intake, protein content, bone content, maintenance energy requirement, mature size, hide quality, pattern of fat deposition, ribeye area, and ovulation rate.

In some embodiments, the system 10 can perform a method for managing animals at a facility configured for managing bovine subjects. The management component 200 can obtain information from the genetics component 300 that identifies a trait for a first bovine subject of the bovine subjects. The information obtained by the management component 200 can be inferred by analyzing a biological sample of the first bovine subject. The measurement component 100 can determine a physical characteristic of the first bovine subject using imaging and communicate this information to the management component 200, which can manage the first bovine subject at the facility based on at least the identified trait and the determined physical characteristic.

In one embodiment, the system 10 can determine a marbling value for the first bovine subject using imaging (e.g., by an ultrasound measurement taken by the measurement component 100) and a marbling genetic value based on analysis of a sample from the first bovine subject. The management component 200 of the system 10 can sort the first bovine subject into one of multiple predefined groups based on at least the obtained marbling genetic value and the determined marbling value from imaging.

This method also includes managing animals with a measurement component of an internal characteristic of the animal that is determined using imaging. The imaging may be carried out using an imaging measurement component 126 that is controlled by the physical measurement control 120. Imaging can include any or all techniques for obtaining an image of an animal or a part of it. Accordingly, the imaging measurement component 126 can be configured for performing any or all of the measurement techniques mentioned above.

In some embodiments, the system 10 can perform a method for managing animals in which the genetics component 300 obtains genetic information regarding a first bovine subject determined by identifying, in a biological sample from the first bovine subject, at a panel of SNPs. For example, the panel of SNPs can be directed to positions that are about 500,000 or less nucleotides from position 300 of at least one of SEQ ID NOS:19473 to 21982. The management component 200 of the system 10 can generate a future weight estimate and a future backfat estimate for the first bovine subject using at least one physical measurement of the first bovine subject, taken by the measurement component 100, and an equation configured to make estimations for a single animal to provide a score for the animal. The management component 200 can use the outputted score to manage the first bovine subject based on the genetic information, the future weight estimate, and the future backfat estimate.

In particular implementations, animals are brought to a feedlot with the expectation that they will later be shipped from the feedlot to a beef packing plant for slaughter. The exact length of time that each animal will spend at the feedlot has typically not been determined when the animal arrives. Rather, the specific shipping date can be determined while they are at the feedlot.

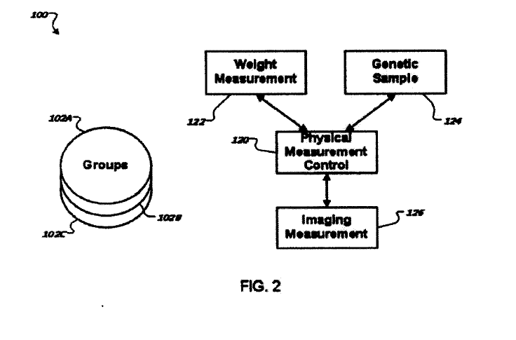

Referring to FIG. 2, groups 102 of animals at the feedlot may be subjected to external physical measurements (e.g., weighing), internal imaging (e.g., ultrasound), and biological sampling (e.g., taking a sample, such as blood, that contains nucleic acid for genetic characterization). These tests can be performed immediately upon arrival of one or more individual animals to the feedlot, or at any other time. A physical measurement control 120 may communicate with and/or control a weight measurement component 122, including a scale that can weigh animals (e.g., those belonging to the groups 102). In addition to the identification and weighing, upon arrival, the physical measurement component 120 can cause a biological sample 124 to be taken from each of the individual animals (e.g., a blood sample, a tissue sample, or the like) to be processed by the genetics component 300. This sample can be processed on location, or sent out to a laboratory elsewhere, for the purpose of identifying certain genomic information (e.g., nucleotide sequences) that correspond to traits such as marbling, average daily weight gain, fat thickness, tenderness, and the like. Based on the results obtained by testing the biological sample, an individual animal can be given a score indicating its predicted ability to efficiently gain weight, and/or produce meat with favorable qualities such as marbling, tenderness, and the like. The identification of genomic information and/or scoring may be carried out using the genetic component 300.

Identification, weighing, imaging, and biological sampling may be carried out while the animals are processed through a chute or other device that temporarily restricts the animal's movement. An individual animal record that can include data such as arrival weight is established in the system at this time. Also, at the time of arrival or later, the individual animals can be grouped and/or categorized. This grouping can be based on the external physical measurements of the animal (e.g., weight) taken at the time of arrival and/or the internal characteristics of the animal (e.g., results of the imaging measurement 126). The group that an animal is assigned to can indicate management choices such as an expected amount of time that the animal is going to be fed before going to harvest, the type of food and/or additives to feed to the animal, and/or the type of initial implant that the animal is to receive. In some implementations, each animal is classified into one of Early, Normal and Extended categories, for example by registering the categorization in a computer. Such assigned categories can be taken into account in sorting, or other management decisions regarding an animal.

In some embodiments, an optional implant of medicaments and/or compositions can be performed. This can, for example, cause the individual animals to grow faster and/or more efficiently. In other embodiments, the same type of implant is given at arrival to all animals, or all animals in the same category, and is not determined using information from the biological sample. In some instances, genetic or physical characteristic measuring may identify a group of animals that will receive an implant.

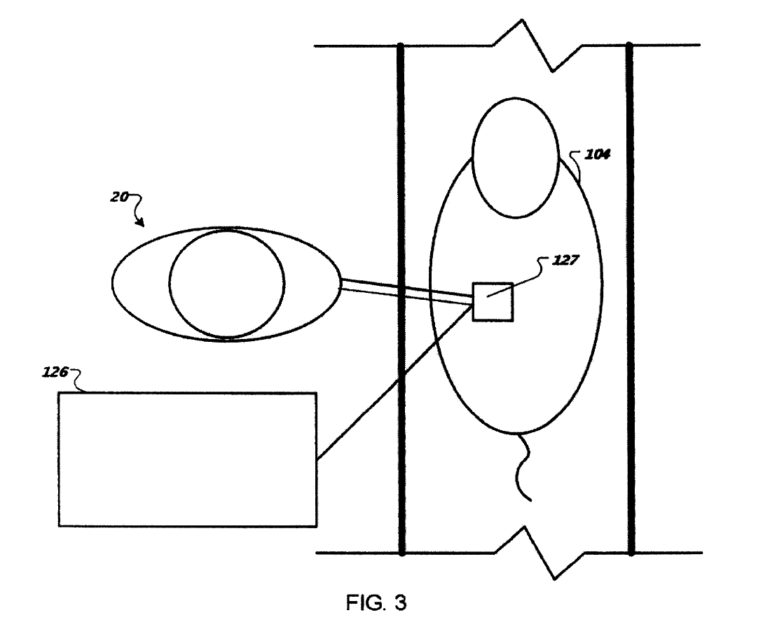

FIG. 3 shows an exemplary top view of an operator 20 making an ultrasound measurement using the imaging measurement component 126. The operator 20 is measuring an animal 104 that is located in a processing chute 106. The imaging technique used in this example is ultrasound, and the operator therefore applies a handheld ultrasound transducer 127 to a particular location on the animal 104 to make one or more measurements. The transducer 127 is connected to the imaging measurement component 126, which registers the measurement(s) for use in the system 10.

In implementations where one or more other imaging techniques are used, the operator 20 could use multiple imaging techniques, to capture one or more images of the animal. The image information can then be processed in a suitable way to obtain the desired measurement. In one embodiment, an MRI image, an x-ray image or a photograph can be automatically processed to obtain one or more numerical values. It is possible for several different characteristics to be measured using imaging. Using the individual animal identification, this information is stored in the system 10 in association with, for example, the original weight of the individual animal and/or any obtained genetic information.

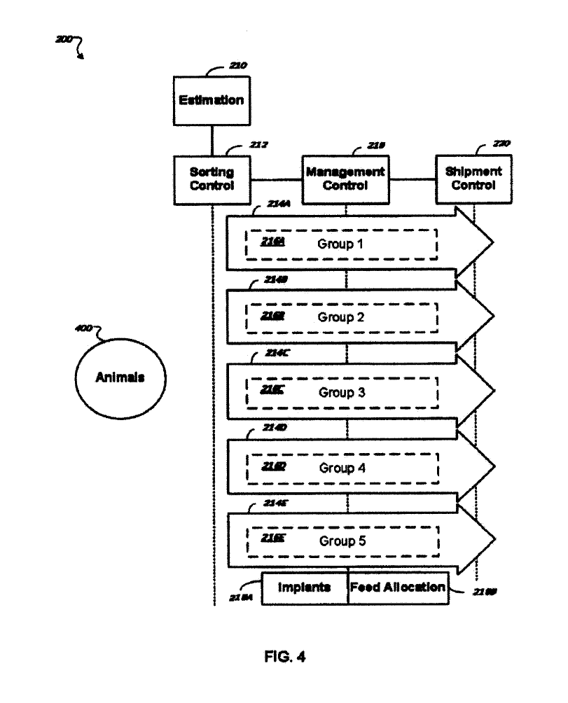

A method of sorting animals into groups at the feedlot will be described with reference also to FIG. 4. An estimation component 210 can make calculations based on data obtained about individual animals. The estimation component 210 can be included in the management component 200. The calculations may involve using equations configured to make estimations or predictions. For example, the estimation component 210 may generate predictions such as a marbling score, a future weight estimate, and/or a future backfat estimate for each of the animals using at least one of the physical measurements, results of the imaging, and/or genetic information from the animal. The estimation component 210 may do so by inserting the physical measurement(s), imaging results, and/or genetic values, into an equation that is configured to make estimations for a single animal. That is, the estimations and predictions can be made on an individual animal basis while management of animals in the system 10 can be done on a group basis. The animal can be sorted into one of several groups based on the scores using these equations, as will be described. The estimation component 210 may perform the calculations while the animal is captured in the processing chute 106, or as an alternative, the calculations can be performed at a later time (e.g., after additional processing, after obtaining results of genetic sampling, and the like).

Data that the estimation component 210 may use in the calculations includes, but is not limited to: initial weight, date of initial weight, current weight, date of current weight, expected average days to market for the group, imaging backfat, imaging muscle tissue depth (e.g., ribeye depth), imaging marbling, marbling genetic value, average daily gain genetic value, past and future implant strategy, and breed code. Data that the estimation component 210 may generate based on the calculations includes, but is not limited to: days fed, average daily gain to date, days to feed, estimated future feed intake, estimated future average daily gain, estimated weight at future dates, estimated backfat at future dates, estimated marbling at future dates, food additive regimen, and implant strategy.

In some embodiments, some or all of the estimations and predictions generated by the estimation component 210 may be used by a sorting control component 212 in the system 10 to categorize individual animals. The operations performed by the sorting control component 212 include passing the estimations and predictions through a series of logical tests to make categorization decisions. The sorting control component 212 provides a signal representative of the categorization decision. This signal can then be used to assign individual animals to sort groups.

For example, the categorization decision may include assigning each animal to one of several predetermined groups. In some embodiments, categorization can occur substantially immediately after testing is performed on an individual animal. In other embodiments, the sorting control component 212 can perform the categorization whenever prompted by the system 10 or by the operators of the system 10. An individual animal can be grouped based on, for example, the current and past physical measurements (e.g., weight measurements 122) of the animal, the internal characteristics of the animal (e.g., results of the imaging measurements 126), and the results of the genetic testing (e.g., as performed by the genetics component 300). In some embodiments, the group that an animal is placed in can indicate an expected amount of time that the animal is going to be fed before going to harvest, can indicate the type of food and/or additives to feed to the animal, and can indicate the type of implant that the animal is to receive. This information can be added to the individual record of the animal that was established upon arrival.

In one embodiment, for time spent in a feedlot, three categories are used to sort incoming animals: early, normal and extended. In one embodiment, the early category generally refers to 20-80 days feeding period prior to harvest, although in another embodiment, this time period is between 35-75 days feeding prior to harvest. The normal category generally refers to 70-130 days feeding prior to harvest, although in another embodiment, this time period is between 80-105 days feeding prior to harvest. The extended category generally refers to 90-160 days feeding prior to harvest, although in another embodiment, this time period is between 110-150 days feeding prior to harvest. One of ordinary skill in the art will recognize that these times are relative and may be adjusted as animal management capabilities improve. As time ranges are adjusted, the early period will precede the normal time period which will in turn precede the extended period.

In some embodiments, one or more lots of animals 400 can be subjected to processing by the system 10 in FIG. 4. At this time, the animals 400 can be in a common group that has been kept together at the feedlot for some time (optionally the animals in such a common group can earlier have been categorized into the Early, Normal and Extended categories). As another example, the animals 400 that are brought in for processing can come from separate groups in the feedlot. The processing can include physical measurements, imaging, and the like, for example as described earlier. The animal is identified so that the previous history of the animal is available and so that the results of the current processing can be added to the animal's record.

The genetic information obtained from the biological sample (taken at an earlier processing stage) may have been obtained before the processing described above. For example, a certain time may be necessary to forward the sample to a lab, perform the analysis there, and return the results. The genetic information can therefore become available sometime in between the biological sampling and the current processing. On the other hand, in an implementation where genetic information is obtained more rapidly (such as by performing the nucleotide analysis at the animal management facility), the time of taking the biological sample or performing the sorting based on genetics, or both these measures, can be adjusted differently.

The estimation component 210 may generate predictions such as a marbling score, average daily gain, a tenderness score, a future weight estimate, and/or a future backfat estimate, for each of the animals using at least one of the physical measurements, the results of imaging, and/or genetic information from the animal. The estimation component 210 may do so by inserting the physical measurement(s), imaging result(s), and/or genetic values into an equation that is configured to make estimations for a single animal. That is, the estimations and predictions can be made on an individual animal basis while management of animals in the system 10 can be done on a group basis. The estimation component 210 may generate estimates of the average daily weight gain and/or the quality of meat at harvest (e.g., marbling, tenderness, and the like) of an individual animal based on factors such as traits (i.e., phenotypes) of the animal and additional factors, for example, but not limited to, past and future implant strategy, marbling determined by imaging, and estimated backfat at harvest. To facilitate the calculations performed by the estimation component 210, the results of the analysis of the genetic material obtained upon arrival may be reduced to numerical values (e.g., molecular genetic values or MGVs), that represent certain traits, such as marbling and/or average daily weight gain, of the animal. The estimation component 210 may generate more than one estimate based on varying certain independent variables such as future implant strategy and number of days until harvest, thus creating a family of estimates based on variations in the independent variables. The estimation component 210 may perform the calculations while the animal is captured in the processing chute 106.

The animal can be sorted based on the results of the calculations. For example, the animals can be sorted into sort groups 216 that are distributed among pens 214. Such sorting can be effectuated by the sorting control 212. Additional animal management can be provided by a management control 218, and the shipment of the animals can be controlled by a shipment control 220. These components will be described further below.

The following examples will serve to further illustrate the present invention without, at the same time, constituting any limitation thereof. It is to be clearly understood that resort may be had to various embodiments, modifications, and equivalents thereof which, after reading the description herein, may suggest themselves to those skilled in the art without departing from the spirit of the invention.

### EXAMPLES

### Example 1

### Estimating Empty Body Fat of a Live Animal

Empty Body Fat (EBF) of the live animal is estimated using the current weight and imaging measures. The estimate of EBF is employed in calculation of future gain, future weight, and future backfat. Another aspect of the calculations is to estimate the future weight of an individual animal. The future weight of an animal is determined from, for example, an estimated daily-gain-to-finish measure and/or information related to the animals ability to gain weight, such as, an average daily gain (ADG) prediction, an ADG molecular genetic value (ADG MGV), and the like. Because it is typically desirable to harvest individual animals at a particular weight, the estimation component 210 can estimate a days-to-critical-weight measure for an individual animal based in part on data such as a predefined critical weight for the animal and/or an ADG prediction. The days-to-critical-weight measure is a numerical value indicating the number of days that an individual animal must remain in a feedlot to attain a predetermined weight and can be used in the sorting of animals into predefined groups.

### Example 2

### Estimating Marbling of an Animal

Marbling of the animal at the time of harvest, which is important in determining an animal's ability to produce meat that will receive a high grade at harvest (i.e., USDA Choice or Prime) is estimated. Data used to estimate marbling includes data derived from the results of testing genetic material (e.g., the marbling molecular genetic value, MMGV), the chosen implant strategy (e.g., type and amount of medicaments and/or compounds provided), the marbling determined by imaging, and the estimated backfat at harvest. For example, animals with a high MMGV can have a tendency to produce meat that will grade higher than meat from animals with a low MMGV. However, the administration (e.g., implanting) of, for example, estrogenic and androgenic compounds that are intended to encourage efficient weight gain and/or addition of lean muscle mass can have a negative impact on animals ability to produce meat that will receive a high grade. Thus, equations can be used to predict the effect of implant strategies (i.e., types and amounts of implanted compounds) on an individual animal's ability to produce meat that will grade.

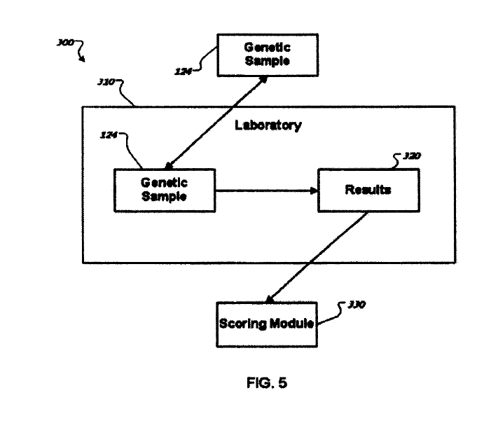

The marbling molecular genetic value (MMGV) is used in the calculations that are performed by the estimation component 210. The MMGV is determined by the genetics component 300 from information received from a testing facility, such as a laboratory capable of performing identification of SNPs (single nucleotide polymorphisms) from blood or tissue samples. Referring to FIG. 5, a biological sample 124 that was taken from each individual animal under the guidance of the physical measurement control 120 is sent off to a laboratory 310 for genetic testing. The laboratory can be in the same location as the feedlot, or can otherwise be situated at a remote facility specializing in genetic testing. Upon completion of the genetic testing, the results 320 are returned to the genetics component 300 in the form of an electronic copy (e.g., a CD-ROM, floppy disk, USB drive, or the like) and/or can be transmitted electronically to the genetics component 300 via a suitable communications protocol, for example over the internet. Once the results of the genetic testing have been obtained in the genetics component 300, a scoring module 330 of the genetics component 300 analyzes the results and designate a numerical score for certain traits, such as marbling, tenderness, and the like. The scoring module 330 is optionally in the control of the laboratory and can be located on those premises; or can be located elsewhere and receive the results of the analysis.

The MMGV is a numerical score that represents the predicted ability of the animal to produce meat with a high degree of marbling, and thus to receive a high grade at harvest (i.e., USDA Choice or Prime). The larger the numerical value of the MMGV, the better the chance the animal has to produce high quality meat. These values can be passed to the management component 200 for use by the estimation component 210.

Some or all of the estimations and predictions generated by the estimation component 210 may be used by the sorting control component 212 in the system 10. The operations performed by the sorting control component 212 include passing the estimations and predictions through a series of logical tests to make sorting decisions. The sorting control component 212 provides a signal representative of the sorting decision.

### Example 3

### Assigning Animals to Predetermined Sort Groups

Sorting decisions may include assigning each animal to one of the several predetermined sort groups 214. Some sort groups 214 may receive, in addition to their regular feed, a growth promoting beta-adrenergic agonist such as: zilpaterol hydrochloride, which is commercially available as Zilmax® from Intervet, Inc. of Millsboro, Del.; or ractopamine, which is commercially available as Optaflexx™ from Elanco, Inc. of Greenfield, Ind. Other implants include TE-S® or TE-IS®, (VetLife, West Des Moines, Iowa).

In this example, with reference again also to FIG. 4, the system 10 includes five sort groups 214A-E. Each of the sort groups 214 are associated with at least one of the pens 216 in which animals belonging to the sort group are to be kept. The sort groups 214 are associated with a corresponding length of time that the animals are to be kept and fed before harvesting, the type of feed to be given, and/or the type of implant that the animals are to receive. As discussed previously, the feed allocation module 218B of the management control component 218 can manage the feed allocation for each of the sort groups 214 and/or pens 216. The system 10 sorts the animals into five different sort groups to facilitate the group-based management of animals.

Each of the sort groups 214 is associated with a different predefined shipping date and feed additive combination as follows:
The sort group 214A is named “Group 1” for animals that are to be given a standard feed and a shipping date that is early relative to a standard shipping date based on average values, for example about 75 days on feed. Moreover, the animal receives no growth promotant.
The sort group 214B is named “Group 2” for animals that are to be given a standard feed and a shipping date that is early relative to the standard shipping date, but not as early as group 1, for example about 90 days on feed, and the animal receives no growth promotant.
The sort group 214C is named “Group 3” for animals that are to be given a standard feed and a shipping date that is normal relative to the standard shipping date, for example about 105 days on feed, and the animal receives no growth promotant.
The sort group 214D is named “Group 4” for animals that are to be given a standard feed and a shipping date that is extended relative to the standard shipping date, for example about 135 days on feed, and the animal receives no growth promotant.
The sort group 214E is named “Group 5” for animals that are to be given a standard feed supplemented with additional growth promoting compound(s) (e.g., zilpaterol hydrochloride) and a shipping date that is normal relative to the standard shipping date, for example about 105 days on feed.

The predefined shipping dates may be precise, such as 105 days, or may be flexible, such as a 20-40 day interval. Nevertheless, each of the sorting groups are associated with a different combination of shipping dates and feed additives.

The different shipping dates for the respective sort groups are managed by the shipment control component 220 in the system 10. The shipment control component 220 initiates the processing that causes the animals in the pen 216A to be shipped after 75 days on feed. Similarly, it initiates the process of shipping the animals in the pen 216D after 135 days on feed. At or after the time that the animals are sorted into the five sort groups 214, the animals receives an implant of medicament and/or compound that is determined by the system 10, but is not related to the sort groups 214. Exemplary implants include any of TE-S®, TE-IS® (VetLife, West Des Moines, Iowa) and/or Synovex-Plus® (Wyeth, Madison, N.J.). As an additional option, the system 10 may determine that certain animals are not to receive an implant. As discussed previously, the implants are administered by the implants module 218A included in the management control component 218. In some embodiments, the implant strategy is independent of the sort group. Because of this, the implants module 218A are located at the processing chute 106 so that the implants can be made shortly after the sorting decision has been made, but prior to performing the sorting.

The estimation component 210 makes predictions of the likelihood that an animal will produce meat that will obtain grade USDA Choice or Prime. One factor in obtaining these top grades is the amount of marbling present at harvest. To make predictions of the marbling score of an animal at harvest, the estimation component 210 uses information relating to the marbling molecular genetic value (MMGV), the past and future implant dosages, the estimated backfat at harvest, and the marbling and muscle depth at the time of imaging. The genetics component 300 generates an MMGV, a score relating to an animal's ability to produce meat with the correct amount of marbling, from the biological sample 124 of an individual animal. The biological sample 124 is sent to a testing facility substantially immediately after being drawn from an animal and identified in such a way as to be able to trace it back to the individual animal it was drawn from. The testing facility then attempts to identify one or more nucleotide occurrences, which have previously been shown to be associated with a “high” trait characteristic (e.g., correct amount of marbling, a high level of tenderness, ability to efficiently gain weight, and the like). The results of the testing, such as the presence of specific SNP markers, are returned to the genetics module 300. The genetics module 300 reduces the results of the genetic testing to numerical values (e.g., MGVs) that can be used by the estimation component 210 to predict future characteristics of the specific live animal from which the biological sample was obtained.

### Example 4

### Assessing Marbling at Harvest Using Combination MGV and Imaging Information

In some embodiments, a marbling MGV (MMGV) can be used, along with additional data, to quantitatively predict the amount of marbling at harvest of an individual animal. Marbling at harvest (MAH) can be determined according to the following equation:

### Steers

Equation to estimate MAH:

Marbling=128+(0.2877*MGV)−(0.1977*DOSE)+(16.7854*MBLu)+(7.3265*EBFc)

### Heifers

Equation to estimate MAH:

Marbling=63+(0.39*MGV)−(0.10*DOSE)+(12.03*MBLu)+(10.14*EBFc)

### Where

MGV=Molecular Genetic Value for Marbling from BeefGen testing
DOSE=Total hormone dose from all implants
MBLu=Marbling measurement taken with ultrasound
EBFc=Expected Empty Body Fat percentage based on carcass measurements

The marbling molecular genetic value (MMGV) indicates the genetic propensity of an animal to produce meat with a high marbling score. An MMGV of greater than twelve indicates that the animal falls within the top thirty percentile with regards to marbling and has between a 90% and 98% chance to grade (i.e., USDA Choice or Prime) based on a backfat endpoint of 0.5 to 0.6. An MMGV of between −9.5 and 12 indicates an animal that is in the mid thirty percentile (i.e., 30%-60%) with regards to marbling and has between a 45% and 80% chance to grade based on a backfat endpoint of 0.5 to 0.6 and an implant dosage of 14 mg to 158 mg. This indicates that time at the feedlot and implant strategy play a large part in these animals' ability to grade. Finally, an MMGV below −9.5 indicates an animal that is in the bottom 40 percentile with regards to marbling and has only a 10% chance to grade when fed to a backfat endpoint of 0.5 and implanted with a dosage of 158 mg. This category of animal has the least chance to grade and would typically be put on a feeding and implant regimen that would produce the greatest amount of meat with the greatest efficiency without consideration of grade.

By varying the values of BFH (e.g., 0.5 and 0.6) and dose (e.g., 158, 110, and 14), a range of MAHs can be determined for an individual animal. The MAH value which is largest (max MAH) is used in the decision making process of the management control to determine which group the animal should be placed in, what type of feed/additives the animal should receive, and what implants the animal should receive.

The following is an exemplary set of logical tests that can be used to sort individual animals into groups. First, an animal is added to the Group 1 sort group 214A if it was categorized as Early. Animals that are determined to go to Group 1 can be given one of three implants. The animal is given TES® if its MAH is less than 2.45, no implant if its MAH is between 2.45 and 2.52, and in the case where the animal's MAH is greater than 2.52, the animal is given the highest implant dose that results in an MAH that is greater than 2.52 (where TES®>TEIS®>None). The animals in Group 1 sort group 214A will spend a total of 75 days on feed.

An animal is added to the Group 2 sort group 214B if it was categorized as Normal and the animal's MAH is between 1.50 and 2.52. All animals in the Group 2 sort group 214B will spend a total of 90 days on feed and will all receive a TES® implant.

An animal is added to the Group 3 sort group 214C if it was categorized as Normal and has an MAH that is greater than 2.52. The Group 3 sort group 214C will spend a total of 105 days on feed and receive the highest implant dose that results in an MAH that is greater than 2.52 (where TES®>TEIS®>None).

An animal is added to the Group 4 sort group 214D if it was categorized as Extended and has an MAH that is greater than 1.50. Animals that are determined to go to Group 4 will spend a total of 135 days on feed and can be given one of three implants. The animal is given TES® if its MAH is less than 2.45, no implant if its MAH is between 2.45 and 2.52, and in the case where the animal's MAH is greater than 2.52, the animal is given the highest implant dose that results in an MAH that is greater than 2.52 (where TES®>TEIS®>None).

An animal is added to the Group 5 sort group 214E if it passes one of two logical tests. First, the animal is added to the Group 5 sort group 214E if it was categorized as Extended and has an MAH that is less than 1.50. Second, the animal is added to the Group 5 sort group 214E if it was originally as Normal and has an MAH that is less than 1.50. Animals that are determined to go to Group 5 will spend a total of 105 days on feed that is supplemented with a feed additive such as zilpaterol hydrochloride (e.g., based on manufacturer recommendations) and will receive a very high dose implant (e.g., as shown in Table 2).

### Example 5

### Assessing Average Daily Gain Using Combination MGV and Imaging Information

In some implementations, information obtained from a biological sample can be used to infer an individual animals potential for growth. The average daily gain molecular genetic value (ADG MGV), which can be derived from the results of an analysis of a biological sample, can be used by the system 10 to make decisions about the type and amount of implant that an individual animal receives. The presence or lack thereof, of particular SNPs in the biological sample, that can be used to infer an animal's potential for gaining weight, can be used to derive a numerical value (e.g., the ADG MGV). The ADG MGV can be used in a decision making process that determines the amount and type of implant to administer.

### Example 6

### Determining Implant Dose

Table 3 illustrates a decision making process that uses marbling MGV (MMGV) and ADG MGV to determine the implant dose, which is indicated by the terms “High”, “None”, and “Low”. Table 4 lists some exemplary implants and their potencies, while Table 3 lists some exemplary implants and their doses. Animals with marbling MGV greater than 10 can receive a high implant and still be expected to grade Choice. Cattle with marbling MGV lower than −10 also receive a High implant because they would not be expected to grade even if they received no implant. Cattle in the middle column are borderline. Those with reasonably good genetic potential for gain receive no implant in order to give them maximum opportunity to grade. Cattle with low potential for growth get the implant because they are in most need of a growth boost. Table 5 lists examples of total active doses for some implant types.

TABLE 3

| | Marbling MGV (MMGV) | | |
|---|---|---|---|
| ADG MGV | Less than −10 | −10 to +10 | Greater than 10 |
| Greater than 0.625 | High | None | High |
| Between 0.625 and 0.299 | High | None | High |
| Less than 0.299 | High | High | High |

TABLE 4

| Dose | Implant |
|---|---|
| None | No implant given |
| Low | Ralgro ®; Synovex C ®; Component EC ®; Ralgro |
| | Magnum |
| Medium | Synovex ® S, H; Component ® ES, EH |
| Medium High | Finaplix S, H ®; Revalor IS, IH; Synovex ® Choice |
| High | Revalor S, H; Component ® TES |
| Very High | Synovex ® Plus; Revalor ® 200 |

TABLE 5

| | | | Total active |
|---|---|---|---|
| Trade Names | | | dose, mg |
| Ralgro ® | | | 14 |
| Magnum ™ | | | 28 |
| Compudose ® | | | 25.7 |
| Encore ® | | | |
| Synovex-C ®; Implus-C ®; Component | | | 7.2 |
| E-C ® | | | |
| Synovex-S ®; Implus-S ®; Component ® E-S | | | 14.4 |
| Finaplix-S; Component ® T-S | | | 140 |
| Finaplix ®-H; Component ® T-H | | | 200 |
| Synovex ®-H; Implus-H ®; Component ® | | | 14.4 |
| E-H | | | |
| Revalor-G ® | A/E | M | 48 |
| Revalor-S ®; Component TE-S ®; | A/E | H | 144 |
| Revalor ®-H; | | | 158 |
| Synovex ® Plus ™; Revalor ® 200 | | | 220 |

### Example 7

### SNP Marker Panel for Determining Tenderness

A DNA-based genetic test contains a panel of 11 unique DNA markers each on highly associated with expression for tender meat. By measuring the cumulative effects for each of these 11 markers, this test accounts for a substantial proportion of the total genetic variation for this complex metabolic trait.

Since tenderness can only be measured in harvested cattle it is difficult, time consuming and expensive to make genetic progress for this trait using traditional genetic improvement tools. This test allows producers to accurately assess the genetic potential of their breeding stock to produce tender meat. This test also shortens the interval for making genetic progress because it can be used to test animals of any age. Results will allow cattle producers to make early breeding decisions that increase the accuracy of selection and decrease the age at which animals can be selected.

To determine an MGV for the trait of tenderness, a panel of SNP markers is studied where the presence or absence of a SNP at position 300 of 11 sequences allows the operator to infer the trait of tenderness. For this application, the 11 SNP markers used in this tenderness panel is provided in Table 6.

TABLE 6

| Marker | SEQ ID NO: |
|---|---|
| CAPN1x9 | 64924 |
| MMBT07944 | 20614 |
| MMBT25471 | 64923 |
| MMBT05224 | 21645 |
| MMBT11557 | 20221 |
| MMBT07678 | 19885 |
| MMBT09366 | 20559 |
| MMBT19805 | 20427 |
| MMBT21150 | 21108 |
| MMBT07919 | 21499 |
| MMBT07176 | 19861 |

This test has been validated in Angus using samples from the National Carcass Merit Project, representing Angus sires bred to Angus-based commercial cows. While this is a small population of animals, the data indicate that this test accounts for 100% of the genetic variation observed in this population as measured by Warner-Bratzler shear force. Results of this validation are presented in Table 7.

TABLE 7

| | |
|---|---|
| No. of samples | 407 |
| Heritability* | 0.35 |
| No. of Markers | 11 |
| Phenotypic variation explained (R²)** | 0.38 |
| As a percent of Heritability | 100% |

*as estimated in Minick et al, 2005, Cam. J. Anim. Sci. 84: 599
**estimated from a model that included contemporary group and MGV

A number of embodiments have been described. Nevertheless, it will be understood that various modifications may be made without departing from the spirit and scope of this disclosure. Accordingly, other embodiments are within the scope of the following claims.

What is claimed is:

1. A method for managing animals comprising: obtaining information, at a facility configured for managing bovine subjects, that predicts a trait for a first bovine subject of the bovine subjects, wherein obtaining the information comprises analyzing genetic information in a biological sample of the first bovine subject; and determining a physical characteristic of the first bovine subject using imaging; and calculating a quantitative score based on at least the genetic information and the determined physical characteristic to predict the trait, and managing the first bovine subject at the facility based on the quantitative score.

2. The method of claim 1, further comprising: obtaining, at the facility, the biological sample from the first bovine subject; and forwarding the biological sample to a laboratory to perform the analysis of the biological sample, wherein results of the analysis are received at the facility from the laboratory.

3. The method of claim 2, wherein the results of the analysis are received electronically at the facility from the laboratory.

4. The method of claim 1, wherein managing the first bovine subject comprises performing an operation selected from the group consisting of selecting the first bovine subject for harvest, selecting the first bovine subject for being relocated, selecting the first bovine subject for receiving treatment, selecting the first bovine subject for being measured, selecting the first bovine subject for breeding, grouping the first bovine subject with at least another of the bovine subjects, and combinations thereof.

5. The method of claim 1, wherein the identified trait is at least one characteristic selected from the group consisting of: an average daily weight gain for the first bovine subject, a red meat yield of the first bovine subject, a tenderness of the first bovine subject, an endpoint characteristic of the first bovine subject, a ribeye area of the first bovine subject, and a marbling of the first bovine subject.

6. The method of claim 5, wherein the identified trait that is taken into account in managing the first bovine subject includes at least the marbling of the first bovine subject, further comprising: making an implant decision regarding the first bovine subject based on at least the average daily weight gain for the first bovine subject and the marbling of the first bovine subject.

7. The method of claim 1, wherein the analysis of the genetic sample includes identifying a nucleotide occurrence of at least three SNPs.

8. The method of claim 1, wherein the determined physical characteristic is at least one characteristic selected from the group consisting of: a marbling of the first bovine subject, a backfat measurement of the first bovine subject, a muscle depth measurement of the first bovine subject, and combinations thereof.

9. The method of claim 1, wherein the facility includes an animal management location at which the bovine subjects are to be kept for a yet undetermined time period before being removed therefrom at a shipping date, the method further comprising: receiving the bovine subjects including the first bovine subject at the animal management location, the bovine subjects being organized in several arrival groups; and generating a future backfat estimate for the first bovine subject; wherein managing the first bovine subject comprises sorting, based on at least the future backfat estimate and the identified trait, the first bovine subject into one of several predetermined sort groups for separate management at the animal management location, the predetermined sort groups being different from the arrival groups and associated with different predefined shipping dates.

10. The method of claim 1, wherein the facility is a feedlot where the bovine subjects including the first bovine subject are managed.

11. A method for managing animals, the method comprising: Obtaining a targeted trait genetic value for a first bovine subject based on analysis of a biological sample of the first bovine subject; determining a targeted trait value for the first bovine subject using imaging; and sorting the first bovine subject into one of multiple predefined groups based on at least the obtained targeted trait genetic value and the targeted trait value determined using imaging.

12. The method of claim 11, wherein the targeted trait genetic value includes a marbling genetic value, further comprising: determining a marbling score using the obtained targeted trait genetic value, the determined targeted trait value, an implant dose value for the first bovine subject and a target backfat value for the first bovine subject, wherein the sorting is done based on the marbling score.

13. The method of claim 12, further comprising: selecting one of multiple time categories for the first bovine subject, the categories including at least an early time category, a normal time category, and an extended time category; and determining the marbling score more than once for the first bovine subject while varying at least one of the implant dose value and the target backfat value, the variation being defined in a schedule associated with the selected time category.

14. The method of claim 11, further comprising: selecting an actual implant dose for the first bovine subject based at least on: (i) the targeted trait genetic value; and (ii) a genetic value relating to an average daily weight gain for the first bovine subject, the genetic value based on analysis of a genetic sample of the first bovine subject.

15. A method for managing animals, the method comprising: obtaining genetic information regarding a first bovine subject, the genetic information determined by identifying, in a biological sample from the first bovine subject, at least one single nucleotide polymorphism (SNP); generating a physical attribute estimate for the first bovine subject using at least one physical measurement of the first bovine subject and an equation configured to make estimations for a single animal; and managing the first bovine subject based on the genetic information and the physical attribute estimate.

16. The method of claim 15, wherein managing comprises sorting the first bovine subject into one of several predetermined sort groups for separate management at an animal management location, wherein the predetermined sort groups are different from arrival groups and are associated with different predefined shipping dates.

17. The method of claim 15, wherein the physical measurement is an imaging measurement of the first bovine subject.

18. The method of claim 17, further comprising: estimating an empty body fat measure for the first bovine subject using the imaging measurement; determining a feed allocation using a predefined algorithm taking into account the estimated empty body fat measure; and administering feed according to the determined feed allocation.

19. The method of claim 15, wherein the physical attribute estimate includes a future weight estimate based at least in part on an estimated daily-gain-to-finish measure for the first bovine subject, the estimated daily-gain-to-finish measure also being directly used in managing the first bovine subject; and wherein the managing is also based on an estimated days-to-critical-weight measure for the first bovine subject, the days-to-critical-weight measure being estimated using at least an estimated daily-gain-to-finish measure for the first bovine subject and a predefined critical weight for animals.

20. A method for predicting a trait in an animal subject comprising: analyzing genetic information in a biological sample of the animal subject; and determining a physical characteristic of the animal subject using imaging; and calculating a quantitative score based on at least the genetic information and the determined physical characteristic to predict the trait.

21. (canceled)
